(19) United States
(12) Patent Application Publication
(10) Pub. No.: US 2017/0128177 A1
(43) Pub. Date: May 11, 2017

(54) SPITTOON WITH STERILIZATION AND CLEANING FUNCTIONS AND DENTAL UNIT CHAIR USING THE SAME

(71) Applicant: POINTNIX CO., LTD., Seoul (KR)

(72) Inventor: Joa Rak CHUNG, Seoul (KR)

(21) Appl. No.: 15/318,661

(22) PCT Filed: Jun. 16, 2015
(86) PCT No.: PCT/KR2015/006078

§ 371 Date: Dec. 13, 2016

(30) Foreign Application Priority Data
Jun. 17, 2014 (KR) 10-2014-0073721

(51) Int. Cl. A61C 17/14; A61L 2/26; B08B 9/08; A61G 15/10

(52) U.S. Cl. CPC A61C 17/14; A61G 15/10; A61L 2/26; B08B 9/08; A61L 2202/14; A61L 2202/17; A61L 2202/24

(57) ABSTRACT

A spittoon which is used in a dental unit chair is disclosed. The spittoon includes: a main body; a spitting receptacle disposed in an upper portion of the main body; a spitting receptacle cover configured to cover the spitting receptacle; a moving unit configured to move the spitting receptacle cover, and the moving unit is configured to move the spitting receptacle cover to cover the spitting receptacle or move the spitting receptacle cover not to cover and to open the spitting receptacle.

### FIELD

The present invention relates to a spittoon with sterilization and cleaning functions and a dental unit chair using the same.

### BACKGROUND

A unit chair refers to a chair which is designed to make a diagnosis and treatment for scaling, extracting, or removing teeth, and normally includes a handpiece, a bracket table, a light, a monitor, etc. as a set.

FIG. 1 is a view to illustrate a related-art dental unit chair.

Referring to FIG. 1, the dental unit chair 100 includes a unit chair main body 110, a chair 300, a treatment bracket 200, a spittoon 111, a light 117, a display 180, a holder 130, and an auto anesthesia device 400. Herein, the chair 300 is a tool on which a patient sits or lies to have his/her teeth treated. In this case, the chair 300 is supported on the ground by a base part 315. The unit chair main body 110 is connected with various devices using a plurality of frames 120 and serves to fix various devices.

The light 117 is connected with the unit chair main body 110 via the frame 120 to illuminate an affected area of a patient during treatment.

The display 180 is fixed to the unit chair main body 110 via the frame 120 to display a variety of information provided to a dentist and a patient during treatment.

The treatment bracket 200 is connected with the unit chair main body 110 via the frame 120, and may be provided with an operation panel 121 disposed on the top surface thereof, for operating dental treatment devices and may have various objects necessary for treatment, such as a mouse or the like, placed thereon.

The spittoon 111 is provided with a spitting receptacle and is installed on the unit chair main body 110 in order for a patient to spit out saliva or foreign substances in his/her mouth after or during treatment. Since such a spittoon 111 is used for a patient to spit out saliva including blood or medicine or material used for treatment, and for rinsing patient's mouth, the spittoon 111 should be frequently cleaned. If the spittoon is not frequently cleaned, it becomes dirty and may incur displeasure of patients.

As technology for keeping a spittoon clean, Korean Patent Registration No. 0376400 (Feb. 7, 2005) (titled “Spittoon for Unit Chair”) discloses a spittoon for a unit chair which can be separated and cleaned. The spittoon can be separated and cleaned to be kept clean as disclosed in the above-mentioned Korean Patent Registration, but users may have inconvenience of having to separate and clean the spittoon every time treatment is finished.

### DETAILED DESCRIPTION

### Technical Objects

According to an exemplary embodiment of the present invention, there is provided a spittoon which is hygienic and is easy to sterilize and clean, and a dental unit chair using the same.

According to another exemplary embodiment of the present invention, there is provided a spittoon which is hygienic and can be automatically sterilized and cleaned, and a dental unit chair using the same.

### Technical Solving Means

According to an exemplary embodiment of the present invention, a spittoon used in a dental unit chair includes: a main body; a spitting receptacle disposed in the upper portion of the main body; a spitting receptacle cover configured to cover the spitting receptacle; a moving unit configured to move the spitting receptacle cover, and the moving unit is configured to move the spitting receptacle cover to cover the spitting receptacle or move the spitting receptacle cover not to cover and to open the spitting receptacle.

According to another exemplary embodiment of the present invention, a dental unit chair includes: a spittoon which is used in the dental unit chair, and the spittoon used in the dental unit chair includes: a main body; a spitting receptacle disposed in the upper portion of the main body; a spitting receptacle cover configured to cover the spitting receptacle; a moving unit configured to move the spitting receptacle cover, and the moving unit is configured to move the spitting receptacle cover to cover the spitting receptacle or move the spitting receptacle cover not to cover and to open the spitting receptacle.

### Advantageous Effect

According to an exemplary embodiment of the present invention, the spittoon can be automatically or manually sterilized and cleaned with ease. When a patient does not use the spittoon, the spittoon is covered by a spittoon cover and cleaned, such that the spittoon can be always maintained clean.

### BRIEF DESCRIPTION OF THE DRAWINGS

FIG. 1 is a view to illustrate a related-art dental unit chair;

FIG. 2 is a view to illustrate a spittoon which is used in a dental unit chair according to an exemplary embodiment of the present invention;

FIG. 3 is a view to illustrate a spittoon which is used in a dental unit chair according to another exemplary embodiment of the present invention;

FIG. 4 is a view to illustrate a spittoon which is used in a dental unit chair according to another exemplary embodiment of the present invention;

FIG. 5 is a view to illustrate a spittoon which is used in a dental unit chair according to another exemplary embodiment of the present invention;

FIGS. 6 to 20 are views to illustrate a spittoon which is used in a dental unit chair according to another exemplary embodiment of the present invention;

FIGS. 21 and 22 are views to illustrate a spittoon which is used in a dental unit chair according to another exemplary embodiment of the present invention;

FIGS. 23 and 24 are views to illustrate a spittoon which is used in a dental unit chair according to another exemplary embodiment of the present invention;

FIGS. 25 and 26 are views to illustrate a spittoon which is used in a dental unit chair according to another exemplary embodiment of the present invention;

FIG. 27 is a view to illustrate a movable sterilization device for sterilizing a spittoon which is used in a dental unit chair according to another exemplary embodiment of the present invention;

FIG. 28 is a view to illustrate a movable sterilization device for sterilizing a spittoon which is used in a dental unit chair according to another exemplary embodiment of the present invention; and

FIG. 29 is a view to illustrate a spittoon which is used in a dental unit chair according to another exemplary embodiment of the present invention.

### EXPLANATION OF SIGNS

1, 21, 31, 41, 51, 61: spitting receptacle cover 2: discharge pipe

3: water outlet 4, 14, 24, 34, 54, 64: main body

5: cup 6: first structure

7, 17, 27, 37, 47, 57: spitting receptacle 8: guide bar

9: controller

### BEST MODE

Exemplary embodiments will now be described more fully with reference to the accompanying drawings to clarify aspects, features and advantages of the inventive concept. The exemplary embodiments may, however, be embodied in many different forms and should not be construed as limited to the exemplary embodiments set forth herein. Rather, the exemplary embodiments are provided so that this disclosure will be thorough and complete, and will fully convey the scope of the application to those of ordinary skill in the art.

It will be understood that, when an element is referred to as being “on” another element, the element can be directly on another element or intervening elements. In the drawings, thickness of elements is exaggerated for easy understanding of technical features.

Exemplary embodiments described in the present specification will be described with reference to cross section views and/or plane views which are ideal exemplary drawings of the present invention. In the drawings, thickness of a film and areas are exaggerated for easy understanding of technical features. Accordingly, the forms of the exemplary drawings may be changed according to manufacturing technology and/or an allowable error. Accordingly, the embodiments of the present invention are not limited to illustrated specific forms and include a change in forms caused according to a manufacturing process. For example, an etched area which is illustrated as forming a right angle may be rounded or have a predetermined curvature. Accordingly, areas illustrated in the drawings may have attributes and the shapes of the areas illustrated in the drawings are merely to illustrate a specific shape of an area of an element, and do not limit the scope of the present invention. If the terms such as “first” and “second” are used to describe elements, these elements should not be limited by such terms. These terms are used for the purpose of distinguishing one element from another element only. The exemplary embodiments include their complementary embodiments.

The terms used in the present specification are just to explain the exemplary embodiments and should not be interpreted as limiting the present invention. As used herein, the singular forms “a”, “an” and “the” are intended to include the plural forms as well, unless the context clearly indicates otherwise. It will be further understood that the terms “comprises” and/or “comprising,” when used in this specification, do not preclude the presence or addition of one or more other components.

Hereinafter, exemplary embodiments will be described in greater detail with reference to the accompanying drawings. The matters defined in the description, such as detailed construction and elements, are provided to assist in a comprehensive understanding of the exemplary embodiments. However, it is apparent that the exemplary embodiments can be carried out by those of ordinary skill in the art without those specifically defined matters. In the description of the exemplary embodiment, certain detailed explanations of related art are omitted when it is deemed that they may unnecessarily obscure the essence of the inventive concept.

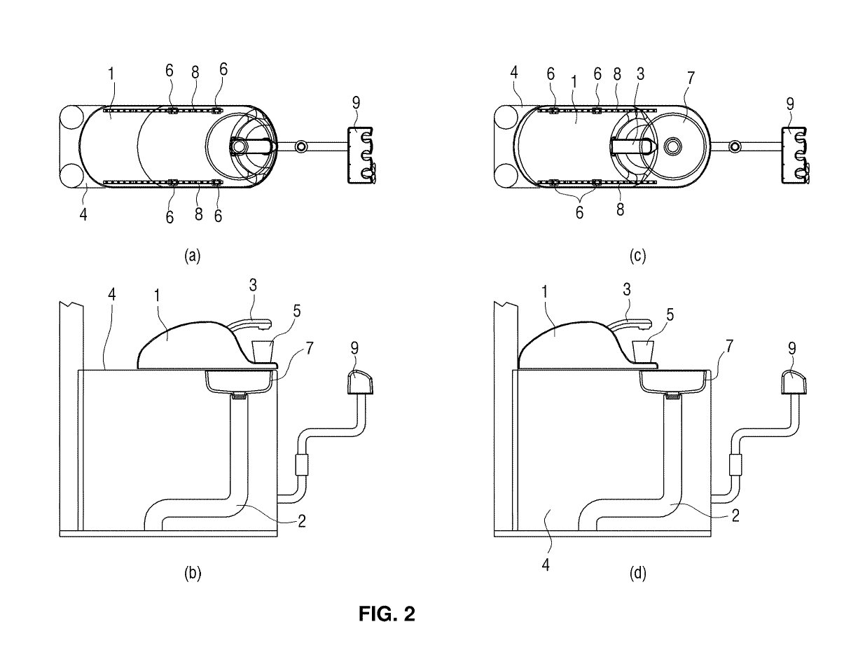

FIG. 2 is a view to illustrate a spittoon which is used in a dental unit chair according to an exemplary embodiment of the present invention.

View (a) of FIG. 2 is a view showing a state in which a spitting receptacle cover 1 covers a spitting receptacle 7 in the spittoon used in the dental unit chair according to an exemplary embodiment of the present invention when being viewed from the above, view (b) of FIG. 2 is a view showing the state of view (a) of FIG. 2 when being viewed from the side, view (c) of FIG. 2 is a view showing a state in which the spitting receptacle cover 1 is moved not to cover the spitting receptacle 7 and to open the spitting receptacle 7 in the spittoon used in the dental unit chair according to an exemplary embodiment of the present invention when being viewed from the above, and view (d) of FIG. 2 is a view showing the state of view (c) of FIG. 2 when being viewed from the side.

Referring to FIG. 2, the spittoon used in the dental unit chair according to an exemplary embodiment of the present invention may include a main body 4; the spitting receptacle 7 disposed in the upper portion of the main body 4; the spitting receptacle cover 1 for covering the spitting receptacle 7; and a moving unit for moving the spitting receptacle cover 1.

According to an exemplary embodiment, the moving unit may move the spitting receptacle cover 1 to cover the spitting receptacle 7 or move the spitting receptacle cover 1 not to cover and to open the spitting receptacle 7.

According to an exemplary embodiment, the moving unit may be configured for a user to manually move the spitting receptacle cover 1 or may be configured to move the spitting receptacle cover 1 by means of a driving means such as a motor. In the embodiment illustrated in FIG. 2, the moving unit is configured for the user to manually move the spitting receptacle cover 1.

According to an exemplary embodiment, the moving unit may include a first structure 6 disposed on the main body; and a guide bar 8 movably connected to the first structure 6. Herein, the first structure 6 may be directly connected with the spitting receptacle cover 1 or may be connected therewith via another structure, for example.

The spittoon used in the dental unit chair according to an exemplary embodiment of the present invention may further include a discharge pipe 2 for discharging saliva including blood, medicine or material used in treatment, or the like, which are contained in the spitting receptacle 7, a water outlet 3 for discharging water (a cleaning liquid) for washing out patient's mouth, and a controller 9 which has an input unit (not shown) for receiving a user's command and controls the operation of the spittoon according to a user's command inputted through the input unit.

It will be easily understood by a person skilled in the art that, in the embodiment of FIG. 2, explanations of elements which are not illustrated and described are omitted since they may unnecessarily obscure the essence of the present invention. For example, the controller 9 may be used to control such elements. Although electronic and mechanical devices are embedded in the main body 4, it will be easily understood by a person skilled in the art that explanations of these devices are omitted since they may unnecessarily obscure the essence of the present invention. In addition, it will be easily understood by a person skilled in the art that, in the other drawings, explanations of elements which are not illustrated and described are omitted unless otherwise specified since they may unnecessarily obscure the essence of the present invention.

Referring to views (a) and (b) of FIG. 2, the spitting receptacle cover 1 according to an exemplary embodiment of the present invention may be moved by the moving unit. When the user holds the spitting receptacle cover 1 with his/her hand and pushes or pulls it in a direction parallel to the guide bar 8, the spitting receptacle cover 1 may cover the spitting receptacle 7. In the present embodiment, the spitting receptacle cover 1 may be directly or indirectly connected with the first structure 6 (which is an element through which the guide bar 8 penetrates and which is movable with the guide bar 8). For example, the first structure 6 may have an annular shape or may have a nut-like shape without a screw thread. When the first structure 6 has the above-described shape, the guide bar 8 may be connected with the first structure 6 by penetrating through the first structure 6. The first structure 6 and the guide bar 8 may be connected with each other to move in relation to each other, and, when the guide bar 8 is fixed and connected to the main body 4 as in the present embodiment, the first structure 6 may be moved along the guide bar 8.

Referring to FIG. 2, when the spitting receptacle cover 1 is moved, the first structure 6 may be moved along the guide bar 8.

Views (a) and (b) of FIG. 2 illustrate a case in which a patient does not use the spittoon by way of an example. That is, when a patient does not use the spittoon, the spitting receptacle 7 may be covered by the spitting receptacle cover 1.

Views (c) and (d) of FIG. 2 illustrate a case in which a patient uses the spittoon by way of an example. That is, when a patient uses the spittoon, the spitting receptacle 7 may not be covered by the spitting receptacle cover 1.

The configuration of the moving unit described above with reference to FIG. 2 is merely an example and the spitting receptacle cover 1 may cover or may not cover the spitting receptacle 7 in other methods.

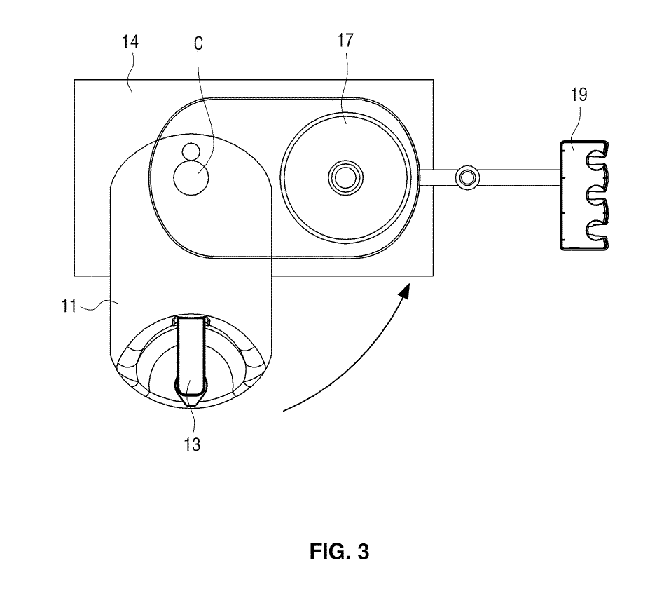

FIG. 3 is a view to illustrate a spittoon which is used in a dental unit chair according to another exemplary embodiment of the present invention.

Referring to FIG. 3, the spittoon having a moving unit mounted therein according to another exemplary embodiment of the present invention is illustrated by way of an example.

Referring to FIG. 3, the spittoon used in the dental unit chair according to an exemplary embodiment of the present invention may include: a main body 14; a spitting receptacle 17 disposed in the upper portion of the main body 14; a spitting receptacle cover 11 for covering the spitting receptacle; and a moving unit for moving the spitting receptacle cover 11. The spittoon according to the present embodiment may further include a water outlet 13 for charging water (or a cleaning liquid).

In the embodiment of FIG. 3, the moving unit may include a hinge C disposed on the main body, and the spitting receptacle cover 11 may be connected with the main body 14 by means of the hinge C and may be rotated about the hinge C.

As shown in FIG. 3, when the spitting receptacle cover 11 is rotated by a predetermined angle in a counter clockwise direction, the spitting receptacle cover 11 may cover the spitting receptacle cover 11. The counter clockwise direction in the present embodiment is merely an example and the spitting receptacle cover 11 may be configured to rotate in the other direction (for example, in a clockwise direction).

In the embodiment of FIG. 3, the spitting receptacle cover 11 may be directly rotated by user's hand. Alternatively, the spitting receptacle cover 11 may be driven and moved by using a driving device.

Regarding elements which have not been described in FIG. 3, please refer to the descriptions of the elements given similar reference numerals in FIG. 2.

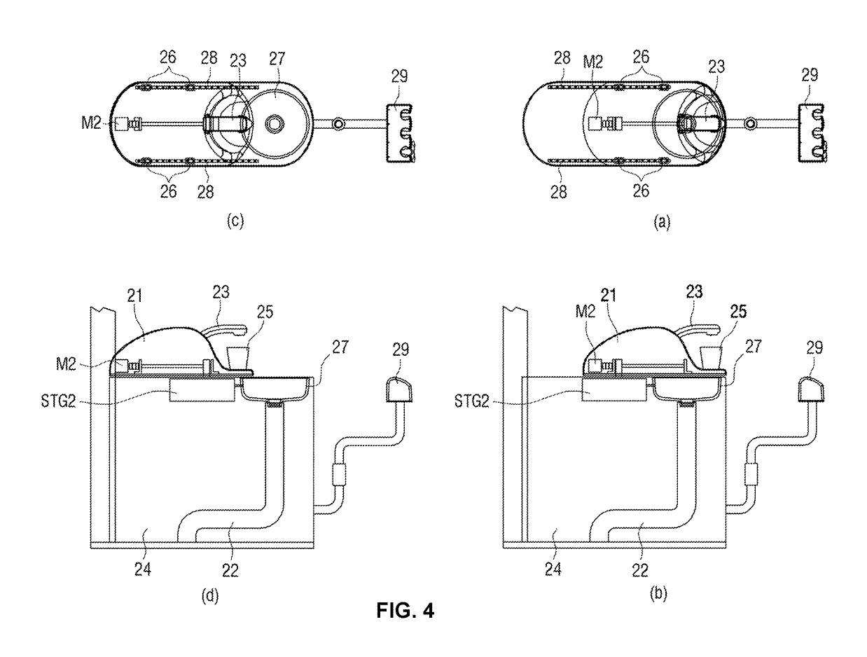

FIG. 4 is a view to illustrate a spittoon which is used in a dental unit chair according to another exemplary embodiment of the present invention.

View (a) of FIG. 4 is a view showing a state in which a spitting receptacle cover 21 covers a spitting receptacle 27 in the spittoon used in the dental unit chair according to an exemplary embodiment of the present invention when being viewed from the above, view (b) of FIG. 4 is a view showing the state of view (a) of FIG. 4 when being viewed from the side, view (c) of FIG. 4 is a view showing a state in which the spitting receptacle cover 21 is moved not to cover the spitting receptacle 27 and to open the spitting receptacle 27 in the spittoon used in the dental unit chair according to an exemplary embodiment of the present invention when being viewed from the above, and view (d) of FIG. 4 is a view showing the state of view (c) of FIG. 4 when being viewed from the side.

The spittoon used in the dental unit chair according to an exemplary embodiment of the present invention may include a main body 24; the spitting receptacle disposed in the upper portion of the main body 24; the spitting receptacle cover 21 for covering the spitting receptacle; a moving unit for moving the spitting receptacle cover 21, and a controller 29.

According to an exemplary embodiment, the moving unit may move the spitting receptacle cover 21 to cover the spitting receptacle 27 or move the spitting receptacle cover 21 not to cover and to open the spitting receptacle 27.

According to an exemplary embodiment, the moving unit may include a first structure 26 disposed on the main body 24; a guide bar movably connected to the first structure 26; and a driving unit M2. Herein, the first structure 26 may be directly connected with the spitting receptacle cover 21 or may be connected therewith via another structure, for example.

For example, the driving unit M2 may include a device like a motor and may be mechanically and/or electronically connected with the spitting receptacle cover 21 to move the spitting receptacle cover 21. When the driving unit M2 is driven, the spitting receptacle cover 21 may be moved along the guide bar 28.

For example, the controller 29 may be provided with an input unit for receiving a command from the user, although it is not illustrated.

For example, when a command for the spitting receptacle cover 21 to cover or open the spitting receptacle 27 is inputted by the user through the input unit, the controller 29 may drive the driving unit M2 to control the spitting receptacle cover 21 to cover or open the spitting receptacle 27.

Views (a) and (b) of FIG. 4 illustrate a case in which a patient uses the spittoon by way of an example. That is, when a patient uses the spittoon, the spitting receptacle cover 21 may not cover the spitting receptacle 27 (that is, may maintain an open state).

Views (c) and (d) of FIG. 4 illustrate a case in which a patient does not use the spittoon by way of an example. That is, when a patient does not use the spittoon, the spitting receptacle cover 21 may cover the spitting receptacle 27.

The spittoon used in the dental unit chair according to the embodiment of FIG. 4 may further include a sterilization module STG2.

According to an exemplary embodiment, the sterilization module STG2 may perform an operation of sterilizing the spitting receptacle 27. For example, the sterilization module STG2 may be configured to perform at least one of the following operations of:
i) generating hot steam and sterilizing by spraying the steam onto the spitting receptacle 27;
ii) sterilizing by spraying hot water (of temperature higher than 60 degrees) onto the spitting receptacle 27;
iii) sterilizing by spraying a compound for sterilizing the spitting receptacle 27 (for example, disinfectant) onto the spitting receptacle 27;
iv) cleaning the spitting receptacle with water; and
v) sterilizing by projecting ultraviolet rays onto the spitting receptacle 27.

For example, the sterilization module STG2 may be configured to perform each of the above-described operations, or to perform at least some of operations i) to v) in sequence and/or simultaneously.

For example, the sterilization module STG2 may be configured to: perform operation i) and then perform operation ii); perform operation i) and then perform operation iii); perform operation iii), perform operation i), and then perform operation iv); perform operation iii) and then perform operation iv); or perform operation i) and operation iv) simultaneously. Alternatively, the sterilization module STG2 may perform one of operations i) to iv) and perform operation v).

The above-described combinations are merely examples and it will be understood by a person skilled in the art that other combinations may be provided.

For example, the sterilization module STG2 may include one of: a sterilizing and cleaning device using steam; a sterilizing and cleaning device using hot water (of temperature higher than 60 degrees); a sterilizing and cleaning device using disinfectant; and a sterilization device using ultraviolet rays, or a combination of the above-described devices.

In the present embodiment, the sterilization module STG2 may be mounted in the main body 24, but this is merely an example and it will be understood by a person skilled in the art that the sterilization module STG2 may be mounted in other locations.

For example, the sterilization module STG2 may sterilize the spitting receptacle 27 when the spitting receptacle cover 21 covers the spitting receptacle 27 (referred to as a close state).

In addition, the sterilization module STG2 may not sterilize the spitting receptacle 27 when the spitting receptacle cover 21 does not cover the spitting receptacle 27 (referred to as an open state).

For example, the controller 29 may control the sterilization module STG2 to sterilize the spitting receptacle 27 in the close state, and control the sterilization module STG2 not to sterilize the spitting receptacle 27 in the open state. To achieve this, an electronic and/or mechanical connection between the controller 29 and the sterilization module STG2 may be configured by a person skilled in the art according to an exemplary embodiment, and thus a detailed description thereof is omitted.

In addition, an electronic and/or mechanical connection of the sterilization module STG2 to sterilize the spitting receptacle 27 may be configured by a person skilled in the art according to an exemplary embodiment, and thus a detailed description thereof is omitted.

The sterilization module STG2 may utilize a steam generation device, a sterilization device using hot water, a sterilization device using a compound, an ultraviolet ray sterilization device, or the like, which are well known in the prior art, and also may utilize devices which will be known after the present application is filed.

Regarding elements which have not been described in FIG. 4, please refer to the descriptions of the elements given similar reference numerals in FIG. 2 or 4.

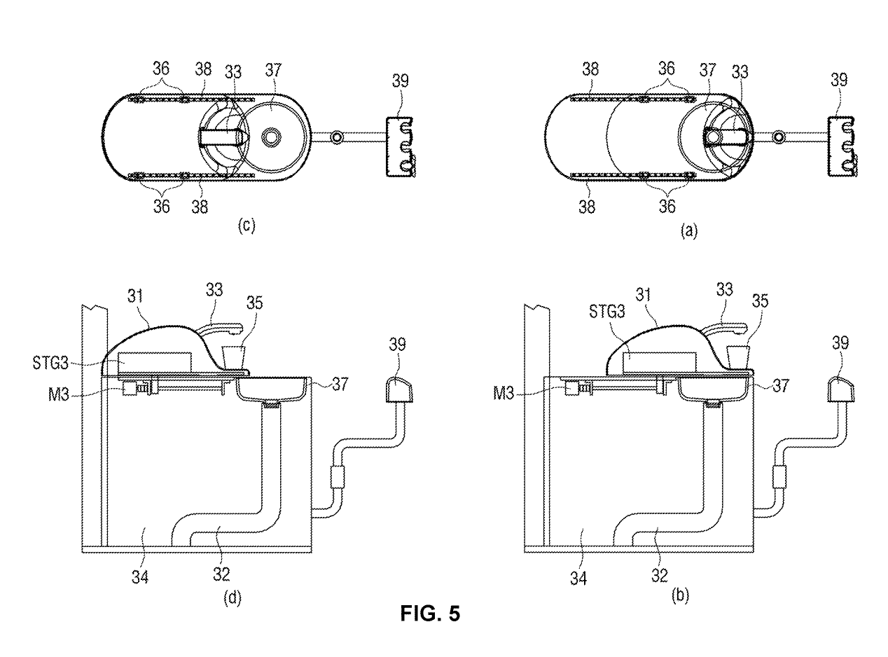

FIG. 5 is a view to illustrate a spittoon which is used in a dental unit chair according to another exemplary embodiment of the present invention.

View (a) of FIG. 5 is a view showing a state in which a spitting receptacle cover 31 covers a spitting receptacle 37 in the spittoon used in the dental unit chair according to an exemplary embodiment of the present invention when being viewed from the above, view (b) of FIG. 5 is a view showing the state of view (a) of FIG. 5 when being viewed from the side, view (c) of FIG. 5 is a view showing a state in which the spitting receptacle cover 31 is moved not to cover the spitting receptacle 37 and to open the spitting receptacle 37 in the spittoon used in the dental unit chair according to an exemplary embodiment of the present invention when being viewed from the above, and view (d) of FIG. 5 is a view showing the state of view (c) of FIG. 5 when being viewed from the side.

The spittoon used in the dental unit chair according to an exemplary embodiment of the present invention may include a main body 34; the spitting receptacle disposed in the upper portion of the main body 34; the spitting receptacle cover 31 for covering the spitting receptacle; a moving unit for moving the spitting receptacle cover 31, and a controller 39.

According to an exemplary embodiment, the moving unit may move the spitting receptacle cover 31 to cover the spitting receptacle 37 or move the spitting receptacle cover 31 not to cover and to open the spitting receptacle 37.

According to an exemplary embodiment, the moving unit may include a first structure 36 disposed on the main body 34; a guide bar 38 movably connected to the first structure 36; and a driving unit M3. Herein, the first structure 36 may be directly connected with the spitting receptacle cover 31 or may be connected therewith via another structure, for example.

The driving unit M3 in the present embodiment is disposed in the main body 34 unlike in the embodiment of FIG. 4.

For example, the driving unit M3 may include a device like a motor and may be mechanically and/or electronically connected with the spitting receptacle cover 31 to move the spitting receptacle cover 31. When the driving unit M3 is driven, the spitting receptacle cover 31 may be moved along the guide bar 38.

For example, the controller 39 may be provided with an input unit for receiving a command from the user, although it is not illustrated.

For example, when a command for the spitting receptacle cover 31 to cover or open the spitting receptacle 37 is inputted by the user through the input unit, the controller 39 may drive the driving unit M3 to control the spitting receptacle cover 31 to cover or open the spitting receptacle 37.

Views (a) and (b) of FIG. 5 illustrate a case in which a patient uses the spittoon by way of an example. That is, when a patient uses the spittoon, the spitting receptacle cover may not cover the spitting receptacle.

Views (c) and (d) of FIG. 5 illustrate a case in which a patient does not use the spittoon by way of an example. That is, when a patient does not use the spittoon, the spitting receptacle cover may cover the spitting receptacle.

The spittoon used in the dental unit chair according to the embodiment of FIG. 5 may further include a sterilization module STG2. The sterilization module STG2 of FIG. 5 differs from the sterilization module STG2 of FIG. 4 in that it is disposed on the top of the main body 34 and is moved with the spitting receptacle cover 31.

According to an exemplary embodiment, the sterilization module STG2 may sterilize the spitting receptacle 37. In the present embodiment, the sterilization module STG2 may be mounted in the main body 34, but this is merely an example and it will be understood by a person skilled in the art that the sterilization module STG2 may be mounted in other locations.

For example, the sterilization module STG2 may sterilize the spitting receptacle 37 when the spitting receptacle cover 31 covers the spitting receptacle 37 (referred to as a close state), but may not sterilize the spitting receptacle 37 when the spitting receptacle cover 31 does not cover the spitting receptacle 37 (referred to as an open state).

For example, the controller 39 may control the sterilization module STG2 to sterilize the spitting receptacle 37 in the close state, and control the sterilization module STG2 not to sterilize the spitting receptacle 37 in the open state.

An electronic and/or mechanical configuration of the controller 39 to control the sterilization module STG2, and an electronic and/or mechanical configuration of the sterilization module STG2 to sterilize the spitting receptacle 37 may be configured by a person skilled in the art according to an exemplary embodiment, and thus a detailed description thereof is omitted.

Regarding elements which have not been described in FIG. 5, please refer to the descriptions of the elements given similar reference numerals in the above-described drawings.

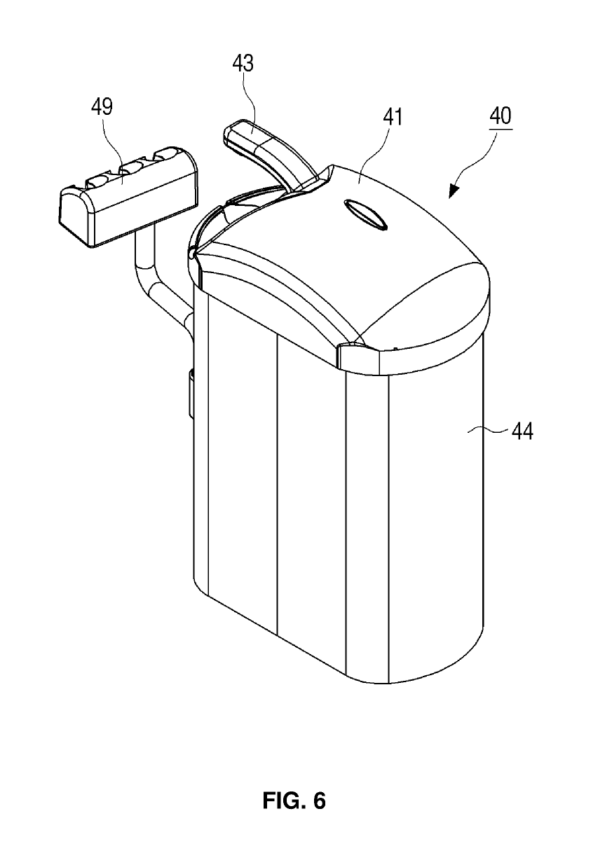

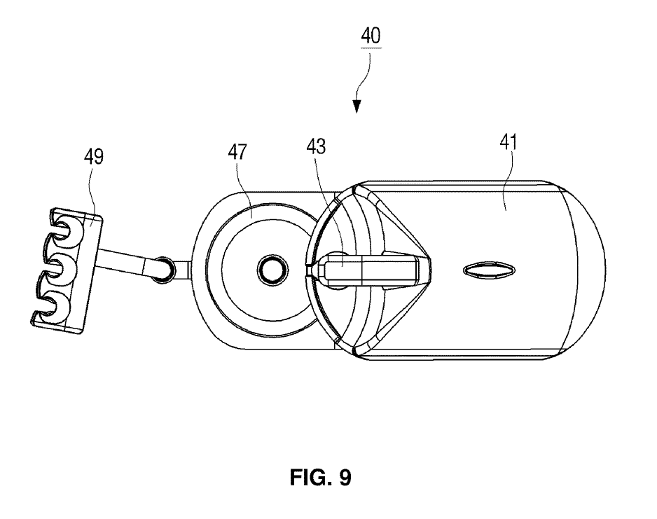

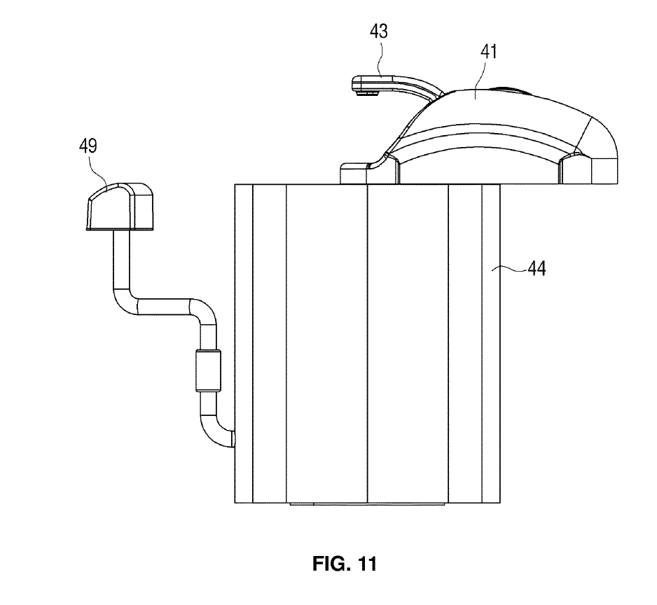

FIGS. 6 to 20 are views to illustrate a spittoon which is used in a dental unit chair according to another exemplary embodiment of the present invention.

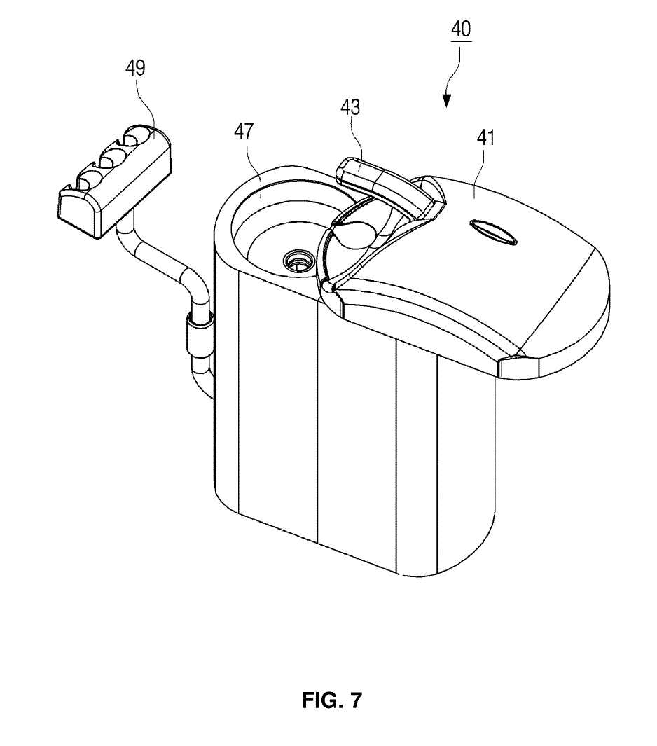

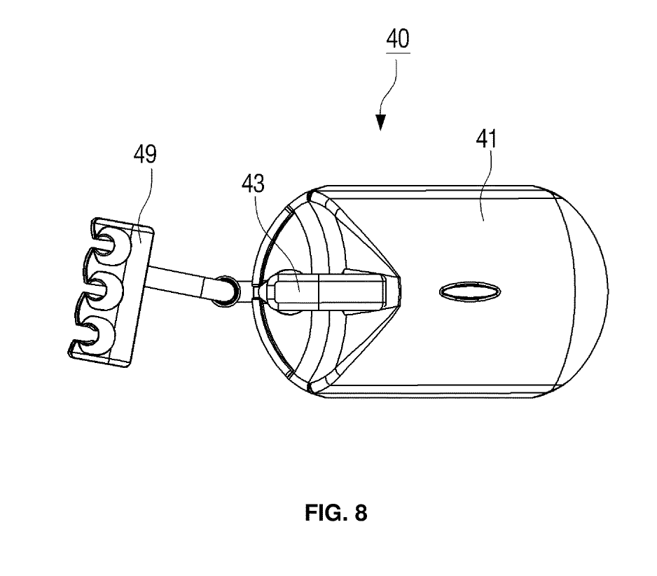

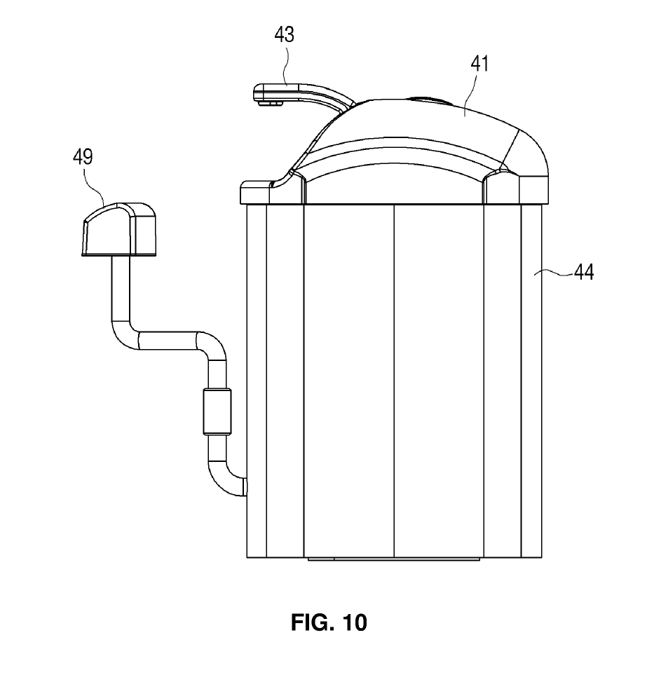

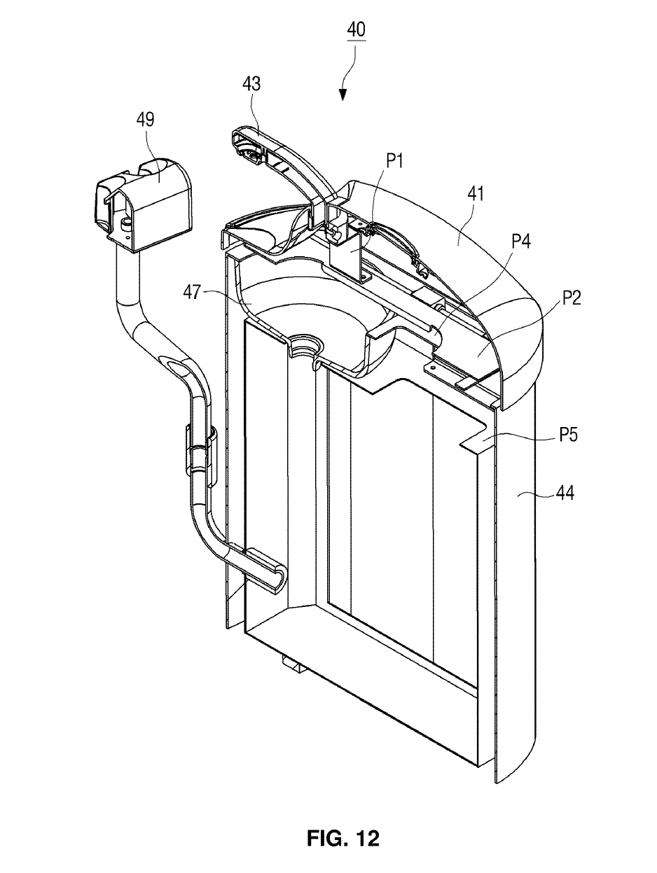

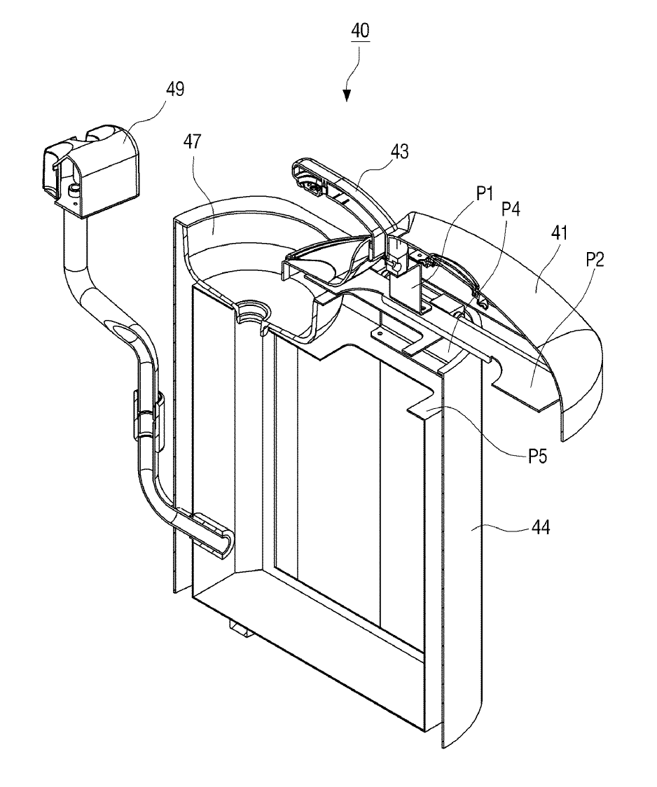

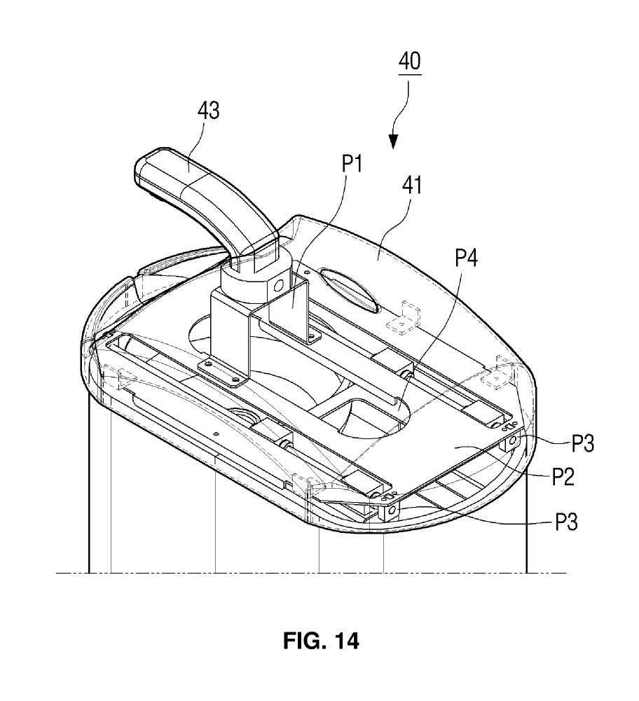

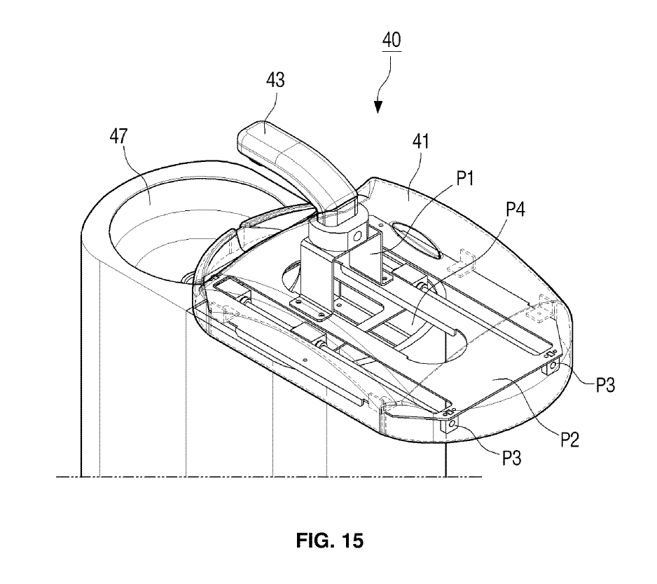

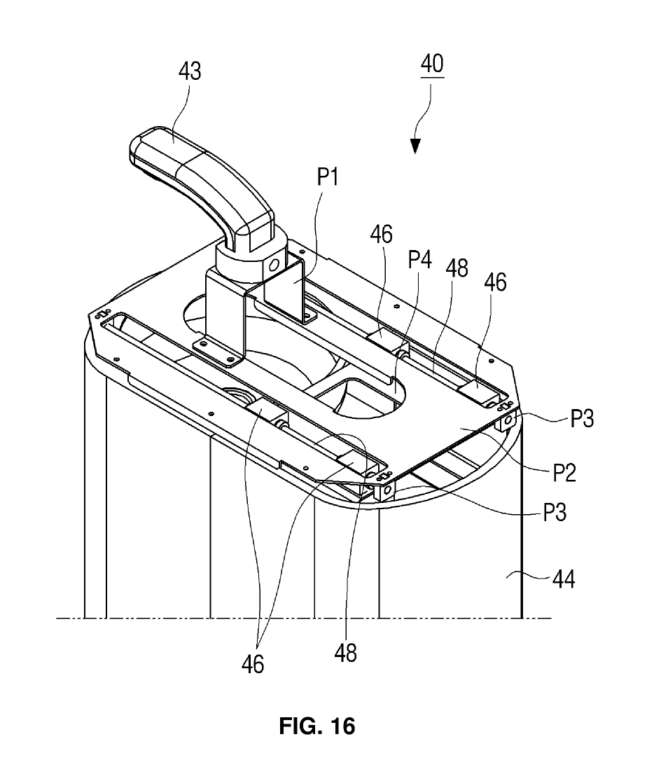

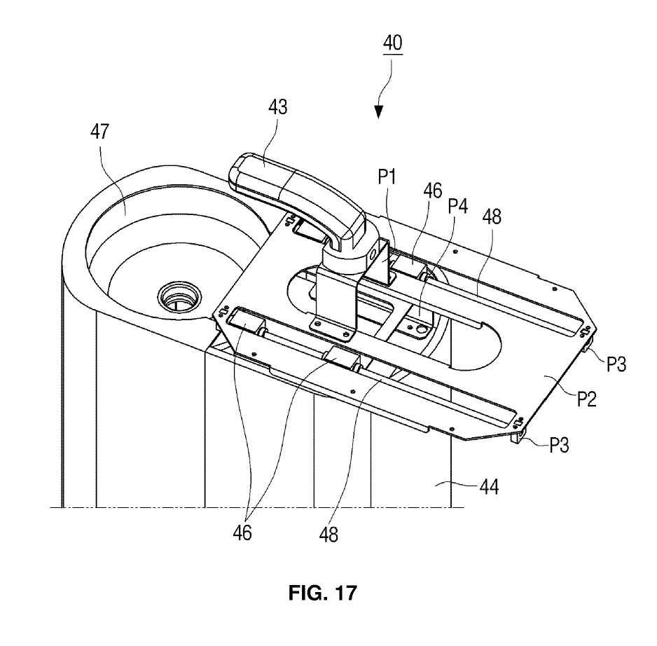

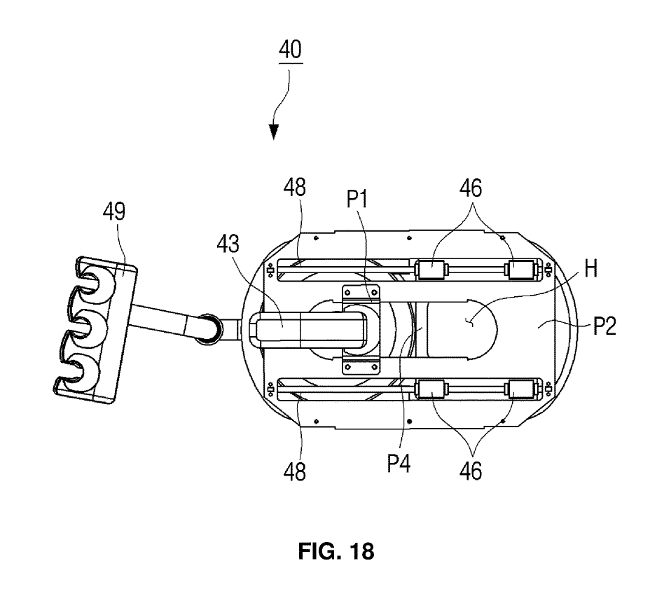

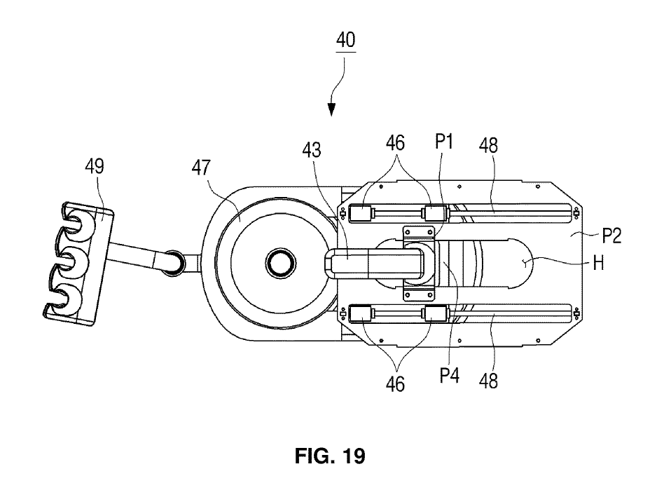

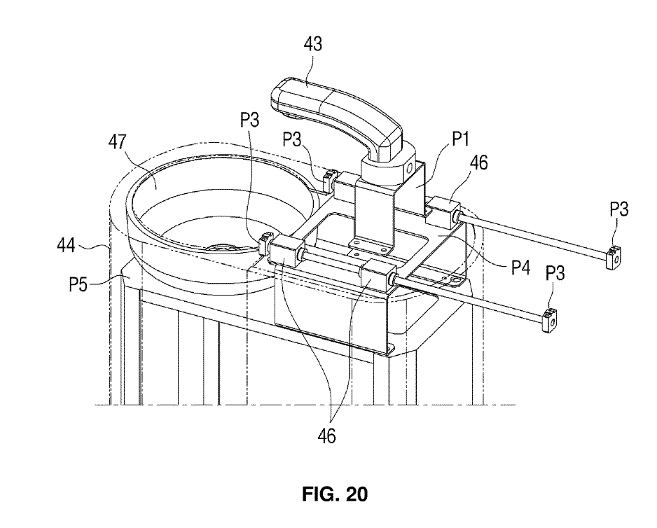

FIG. 6 is a view showing an example of the appearance of the spittoon used in the dental unit chair according to an exemplary embodiment, FIGS. 7, 9, and 11 are views illustrating the spittoon of FIG. 6 in an open state, and FIGS. 8 and 10 are views illustrating the spittoon of FIG. 6 in a close state. FIG. 12 is a cross section view showing the spittoon of FIG. 6 in the close state and FIG. 13 is a cross section view showing the spittoon of FIG. 6 in the open state. In addition, FIG. 14 is a transparent perspective view showing the spittoon of FIG. 6 in the close state and FIG. 15 is a transparent perspective view showing the spittoon of FIG. 6 in the open state. In addition, FIG. 16 is a perspective view showing the spittoon of FIG. 6 from which a spitting receptacle cover 41 is removed in the close state and FIG. 17 is a perspective view showing the spittoon of FIG. 6 from which the spitting receptacle cover 41 is removed in the open state. In addition, FIG. 18 is a plane view showing the spittoon of FIG. 6 from which the spitting receptacle cover 41 is removed in the close state and FIG. 19 is a plane view showing the spittoon of FIG. 6 from which the spitting receptacle cover 41 is removed in the open state. FIG. 20 is a transparent perspective view showing the spittoon of FIG. 6 from which the spitting receptacle cover 41 and a structure P2 are removed in the open state.

These drawings are illustrated as viewed at different angles for the purpose of describing the spittoon used in the dental unit chair according to another exemplary embodiment, and hereinafter, the spittoon used in the dental unit chair according to an exemplary embodiment will be described with reference to the drawings.

Referring to FIGS. 6 to 20, the spittoon 40 used in the dental unit chair according to an exemplary embodiment may include a main body 44, a spitting receptacle 47 disposed in the main body 44, the spitting receptacle cover 41 for covering the spitting receptacle 47, an outlet 43, a moving unit for moving the spitting receptacle cover 41, and a controller 49.

According to an exemplary embodiment, the moving unit may be configured to move the spitting receptacle cover 41 to cover the spitting receptacle 47 or not to cover and to open the spitting receptacle 47.

According to an exemplary embodiment, the moving unit may include a first structure 46, a guide bar 48, a second structure P4, a third structure P2, a fourth structure P1, and a fifth structure P3.

The first structure 46 may be mechanically connected with the guide bar 8, the second structure P4 may be connected with the main body 44, the fourth structure P1 may fix the outlet 43 to the third structure P2, and the fifth structure P3 may fix the guide bar 48 to the third structure P3. The third structure P2 may be mechanically connected with the spitting receptacle cover 41.

It will be easily understood by a person skilled in the art that, in the embodiment of FIGS. 6 to 20, explanations of elements which are not illustrated and described are omitted since they may unnecessarily obscure the essence of the present invention. Although electronic and mechanical devices are embedded in the main body 44, it will be easily understood by a person skilled in the art that explanations of these devices are omitted since they may unnecessarily obscure the essence of the present invention. In addition, it will be easily understood by a person skilled in the art that, in the other drawings, explanations of elements which are not illustrated and described are omitted unless otherwise specified since they may unnecessarily obscure the essence of the present invention.

In the present embodiment described with reference to FIGS. 6 to 20, a sterilization module has not been described. However, the above-described sterilization module may further be included. In this case, the sterilization module may be disposed in the main body 44 or in the spitting receptacle cover 41.

In addition, in the present embodiment described with reference to FIGS. 6 to 20, a driving unit like a motor has not been described. However, a driving unit may further be included. In this case, the driving unit may be disposed in the main body 44 or in the spitting receptacle cover 41.

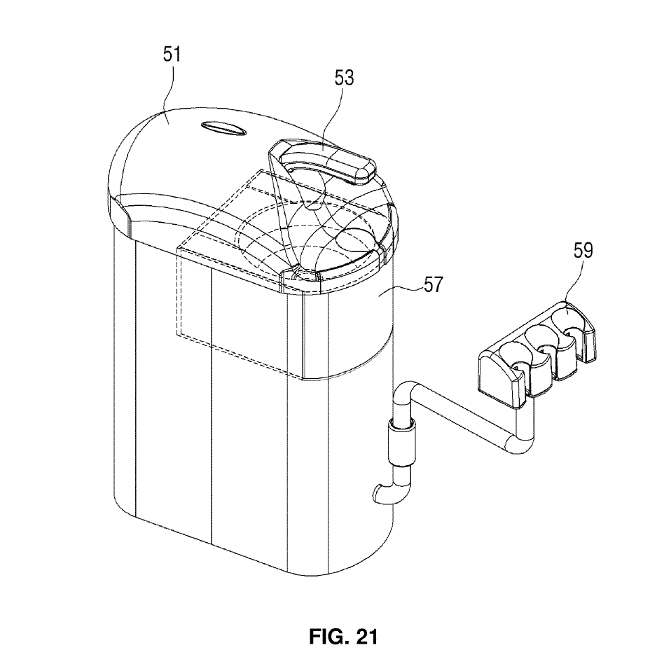

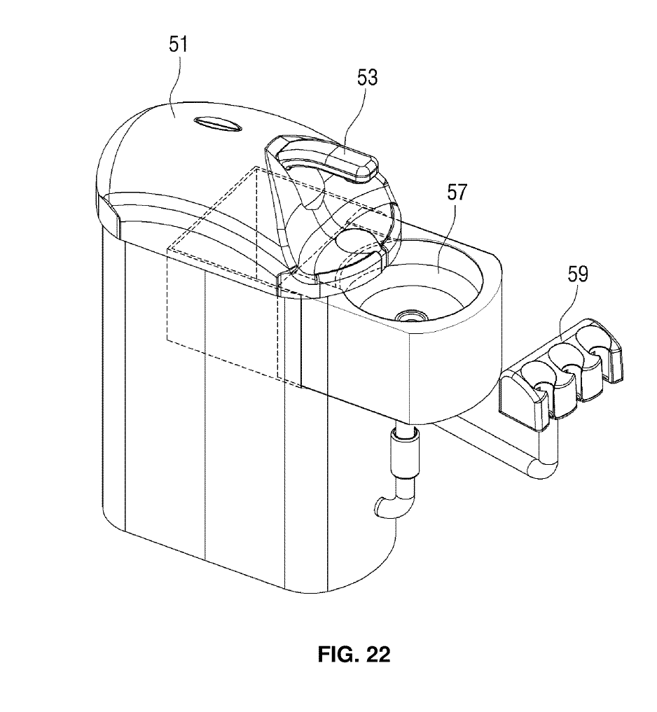

FIGS. 21 and 22 are views to illustrate a spittoon which is used in a dental unit chair according to another exemplary embodiment of the present invention.

Referring to FIGS. 21 and 22, the spittoon according to the present embodiment may include a main body 54, a spitting receptacle 57 disposed in the main body 54, a spitting receptacle cover 51 for covering the spitting receptacle 57, and an outlet 53.

The spittoon according to the present embodiment may have a structure in which the spitting receptacle 57 is movable unlike in the above-described embodiments. For example, in the embodiment of FIGS. 21 and 22, the spitting receptacle 57 may be mounted in the main body 54 and may be exposed to the outside when it is used.

When the spitting receptacle 57 is mounted in the main body 54, the spitting receptacle 57 may be sterilized by a sterilization module (not shown).

The spittoon according to the present embodiment may further include a moving unit (not shown) for moving the spitting receptacle 57, and the moving unit may be configured to mount the spitting receptacle 57 in the main body 54 and expose the spitting receptacle 57 to the outside.

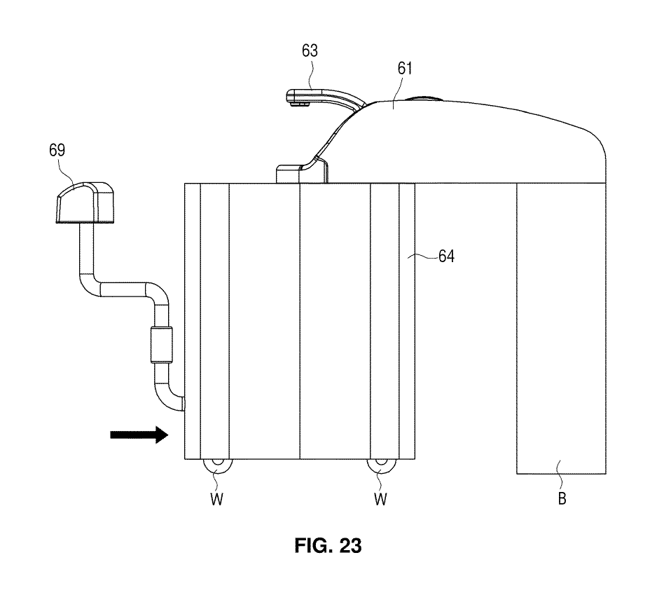

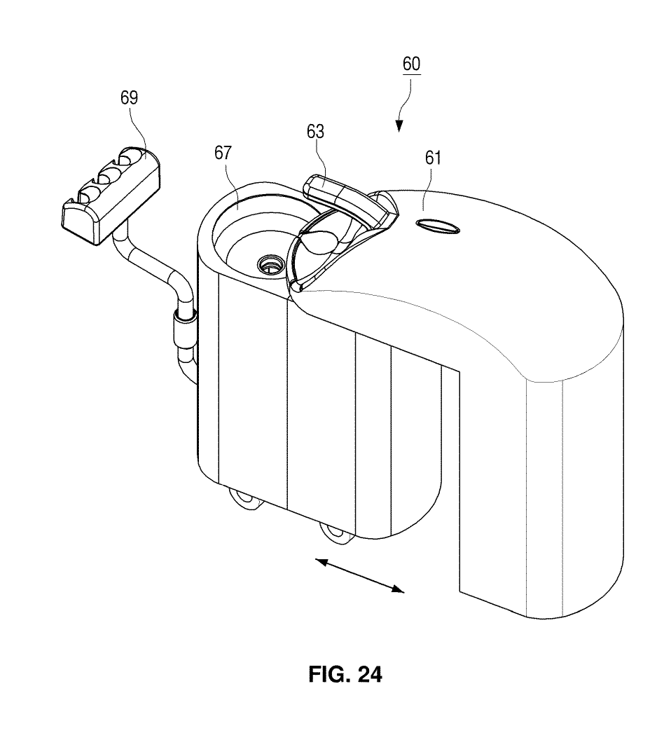

FIGS. 23 and 24 are views to illustrate a spittoon which is used in a dental unit chair according to another exemplary embodiment of the present invention.

Referring to FIGS. 23 and 24, the spittoon according to the present embodiment may include a movable main body 64, a fixed main body B, a spitting receptacle 67 disposed over the movable main body 64, a spitting receptacle cover 61 for covering the spitting receptacle 67, and an outlet 63.

The spitting receptacle cover 61 may be disposed over the movable main body 64 and the fixed main body B.

The movable main body 64 may be provided with wheels (W) and, when the movable main body 64 moves further apart from the fixed main body B, the spitting receptacle 57 enters an open state. On the other hand, when the movable main body 64 is brought in close contact with the fixed main body B, the spitting receptacle 57 enters a close state.

The spittoon according to the present embodiment as described above with reference to FIGS. 23 and 24 differs from those in the above-described embodiments in that the main body has a movable structure. For example, in the embodiment of FIGS. 23 and 24, when the movable main body 64 having the spitting receptacle 67 mounted therein moves further apart from the fixed main body B, the spitting receptacle 67 enters the open state.

When the movable main body 64 is brought into close contact with the fixed main body B, the spitting receptacle 67 may be sterilized by a sterilization module (not shown).

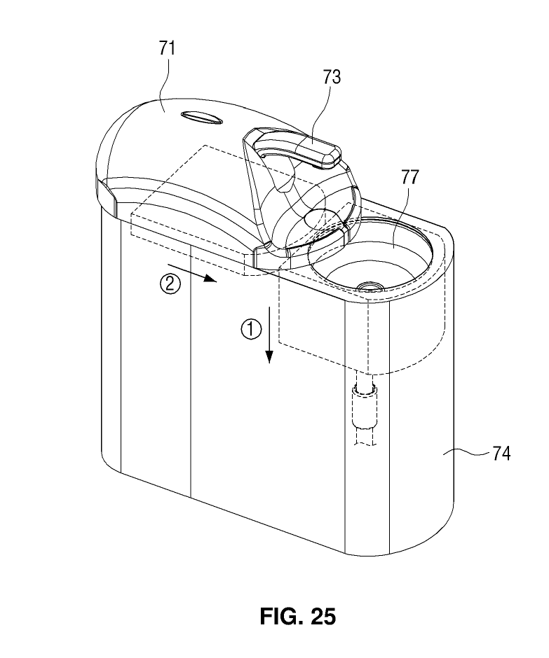

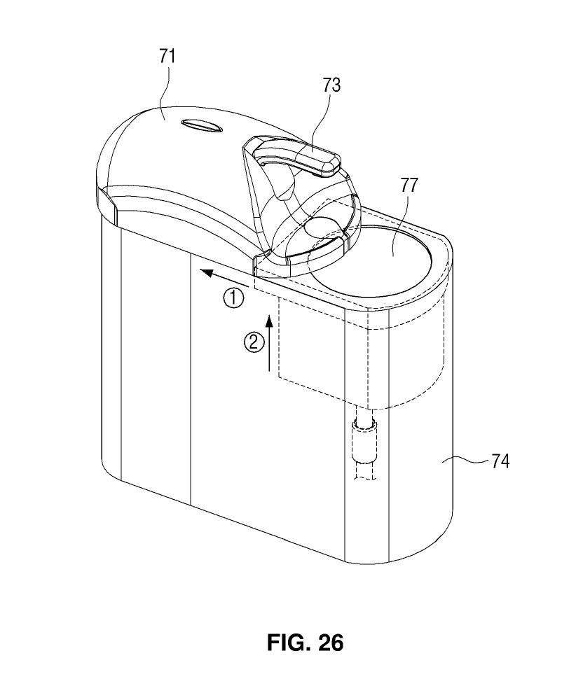

FIGS. 25 and 26 are views to illustrate a spittoon which is used in a dental unit chair according to another exemplary embodiment of the present invention.

Referring to FIGS. 25 and 26, the spittoon according to the present embodiment may include a main body 74, a sterilization module, a spitting receptacle 77 disposed over the main body 74, a spitting receptacle cover 71 for covering the spitting receptacle 77, and an outlet 63.

In the present embodiment, the spitting receptacle 77 and the sterilization module are movable as shown in FIGS. 25 and 26. When the spitting receptacle 77 is not used, the sterilization module may cover the spitting receptacle 77 and sterilize the spitting receptacle 77 as shown in FIG. 26, and, when the spitting receptacle 77 is used, the sterilization module moves not to cover the spitting receptacle 77 as shown in FIG. 25.

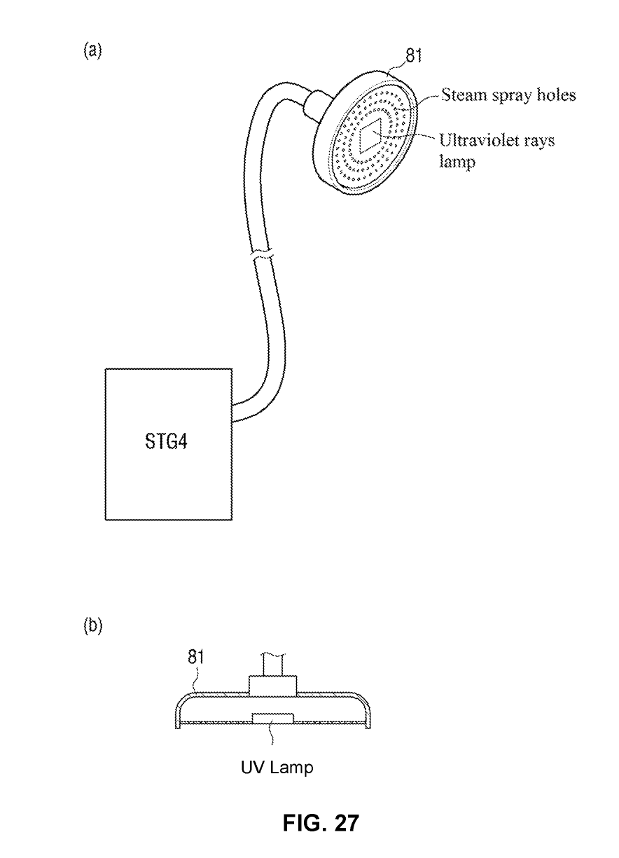

FIG. 27 is a view to illustrate a movable sterilization device for sterilizing a spittoon which is used in a dental unit chair according to another exemplary embodiment of the present invention.

Referring to FIG. 27, the movable sterilization device according to the present embodiment may include a sterilization module STG4, a nozzle, and a spitting receptacle cover 81. The nozzle may include at least one of an ultraviolet ray lamp and steam spray holes for spraying steam. The sterilization module STG4 may have a device for projecting ultraviolet rays and a device for generating steam and spraying steam through the steam spray holes, although they are not illustrated.

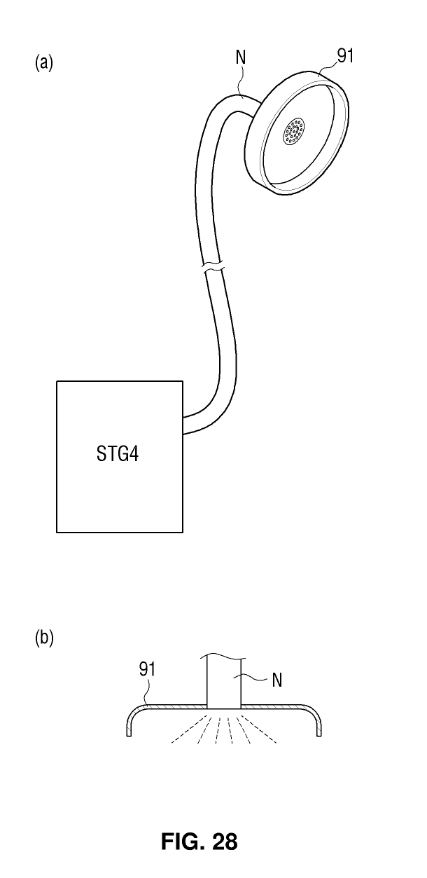

FIG. 28 is a view to illustrate a movable sterilization device for sterilizing a spittoon which is used in a dental unit chair according to another exemplary embodiment of the present invention.

Referring to FIG. 28, the movable sterilization device according to the present embodiment may include a steam generator, a nozzle N, and a spitting receptacle cover 91, and the nozzle may have steam spray holes for spraying stream.

The spitting receptacle cover 91 may be fixed to or removably mounted on the nozzle.

The steam generator may have a device mounted therein, for spraying steam through a nozzle, although it is not illustrated.

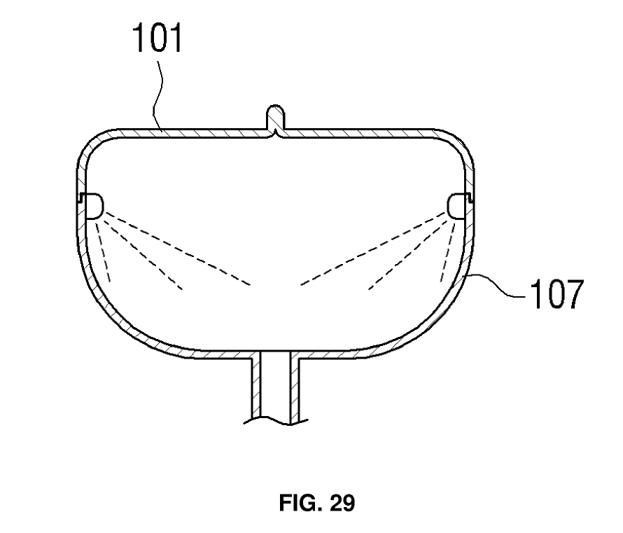

FIG. 29 is a view to illustrate a spittoon which is used in a dental unit chair according to another exemplary embodiment of the present invention.

Referring to FIG. 29, a spitting receptacle 107 used in the dental unit chair according to another exemplary embodiment of the present invention may have spray holes mounted therein, for spraying steam. The spray holes may be formed on one side of the spitting receptacle 107 to spray steam when a spitting receptacle cover 101 covers the spitting receptacle 107. Although not shown, a device for generating steam, which is sprayed through the spray holes, may be mounted in a main body in which the spitting receptacle 107 is disposed, or may be mounted separately from the main body.

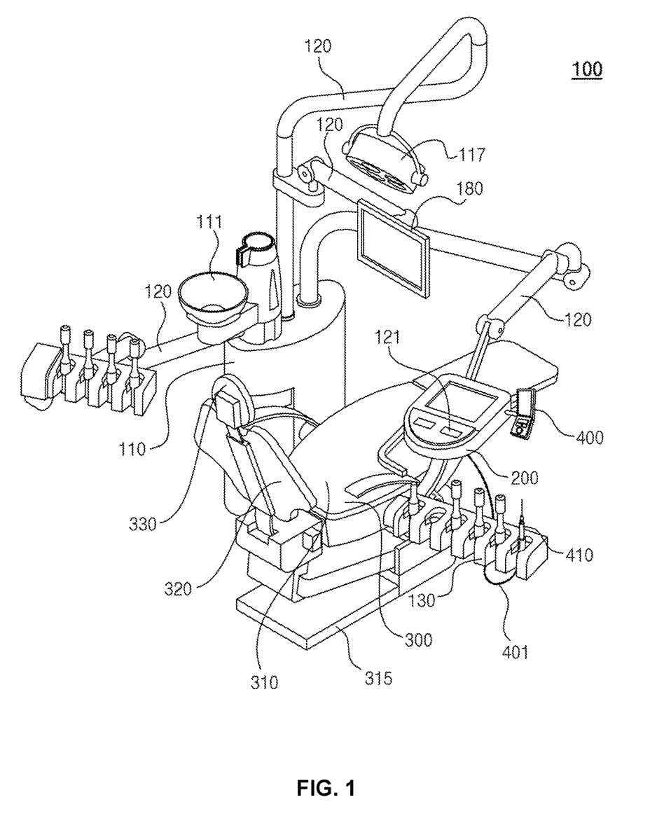

The spittoon used in the dental unit chair according to embodiments of the present invention as described above with reference to FIGS. 2 to 29 may be applied to various types of dental unit chairs. As described above with reference to FIG. 1, the spittoon may be applied to a type of unit chair which is moved with the spittoon or a type of unit chair in which the spittoon is fixed and moved regardless of the unit chair.

While the invention has been shown and described with reference to certain preferred embodiments thereof, it will be understood by those skilled in the art that various changes in form and details may be made therein without departing from the spirit and scope of the invention as defined by the appended claims. Therefore, the scope of the invention is defined not by the detailed description of the invention but by the appended claims.

1. A spittoon which is used in a dental unit chair, the spittoon comprising:
a main body;
a spitting receptacle disposed in an upper portion of the main body;
a spitting receptacle cover configured to cover the spitting receptacle; and
a moving unit configured to move the spitting receptacle cover,
wherein the moving unit is configured to move the spitting receptacle cover to cover the spitting receptacle or move the spitting receptacle cover not to cover and to open the spitting receptacle.

2. The spittoon of claim 1, wherein the moving unit comprises a hinge disposed in the main body,
wherein the spitting receptacle cover is connected with the hinge and rotates about the hinge, such that the spitting receptacle cover covers or does not cover the spitting receptacle.

3. The spittoon of claim 1, wherein the moving unit comprises:
a first structure; and
a guide bar movably connected to the first structure,
wherein the guide bar is directly connected with the spitting receptacle cover or is connected with the spitting receptacle cover via another structure.

4. The spittoon of claim 3, wherein the moving unit further comprises a driving unit, and
wherein the spitting receptacle cover is moved by the driving unit.

5. The spittoon of claim 3, wherein the moving unit further comprises a second structure connected to the main body, and
wherein the first structure is connected to the second structure.

6. The spittoon of claim 5, wherein the moving unit further comprises a third structure P2 which is connected with the guide bar, and
wherein the third structure is directly connected with the spitting receptacle cover or is connected with the spitting receptacle cover via another structure.

7. The spittoon of claim 6, wherein the spittoon further comprises a sterilization module, and
wherein the sterilization module is configured to sterilize and/or clean the spitting receptacle when the spitting receptacle cover covers the spitting receptacle, and is configured not to sterilize and/or clean the spitting receptacle when the spitting receptacle cover does not cover the spitting receptacle.

8. The spittoon of claim 7, wherein the sterilization module is disposed in the main body, or is disposed in the spitting receptacle cover and is moved with the spitting receptacle cover when the spitting receptacle cover is moved.

9. The spittoon of claim 7, wherein the sterilization module is any one of a sterilizing and cleaning device using steam, a sterilizing and cleaning device using hot water, a sterilizing and cleaning device using disinfectant, and a sterilization device using ultraviolet rays, or is a combination of two or more of said devices.

10. A dental unit chair comprising:
a spittoon which is used in the dental unit chair,
wherein the spittoon used in the dental unit chair comprises:
a main body;
a spitting receptacle disposed in an upper portion of the main body;
a spitting receptacle cover configured to cover the spitting receptacle; and
a moving unit configured to move the spitting receptacle cover,
wherein the moving unit is configured to move the spitting receptacle cover to cover the spitting receptacle or move the spitting receptacle cover not to cover and to open the spitting receptacle.

11. The dental unit chair of claim 10, wherein the moving unit comprises a hinge disposed in the main body, and
wherein the spitting receptacle cover is connected with the hinge and rotates about the hinge, such that the spitting receptacle cover covers or does not cover the spitting receptacle.

12. The dental unit chair of claim 10, wherein the moving unit comprises:
a first structure; and
a guide bar movably connected to the first structure,
wherein the guide bar is directly connected with the spitting receptacle cover or is connected with the spitting receptacle cover via another structure.

13. The dental unit chair of claim 12, wherein the moving unit further comprises a driving unit, and
wherein the spitting receptacle cover is moved by the driving unit.

14. The dental unit chair of claim 13, further comprising a controller,
wherein the controller comprises an input unit configured to receive a command from a user, and
wherein, when a command to cover or not to cover the spitting receptacle cover is received from the user through the input unit, the controller may control the driving unit to move the spitting receptacle cover.

15. The dental unit chair of claim 12, wherein the moving unit further comprises a second structure connected to the main body, and
wherein the first structure is connected to the second structure.

16. The dental unit chair of claim 15, wherein the moving unit further comprises a third structure which is connected with the guide bar, and
wherein the third structure is connected with the spitting receptacle cover.

17. The dental unit chair of claim 16, wherein the spittoon further comprises a sterilization module, and
wherein the sterilization module is configured to sterilize and/or clean the spitting receptacle when the spitting receptacle cover covers the spitting receptacle, and is configured not to sterilize and/or clean the spitting receptacle when the spitting receptacle cover does not cover the spitting receptacle.

18. The dental unit chair of claim 17, wherein the sterilization module is disposed in the main body, or is disposed in the spitting receptacle cover and is moved with the spitting receptacle cover when the spitting receptacle cover is moved.

19. The dental unit chair of claim 17, wherein the sterilization module is any one of a sterilizing and cleaning device using steam, a sterilizing and cleaning device using hot water, a sterilizing and cleaning device using disinfectant, and a sterilization device using ultraviolet rays, or is a combination of two or more of said devices.
